(19) United States
(12) Patent Application Publication
(10) Pub. No.: US 2010/0047844 A1
(43) Pub. Date: Feb. 25, 2010

(54) DIAGNOSTIC MARKER FOR FABRY DISEASE

(75) Inventor: Johannes Maria Franciscus Gerardus Aerts, Abcoude (NL)

Correspondence Address: BROWDY AND NEIMARK, P.L.L.C.;624 NINTH STREET, NW, SUITE 300, WASHINGTON, DC, 20001-5303, US

(73) Assignee: Academisch Ziekenhuis bij de Universiteit van Amsterdam, Amsterdam (NL)

(21) Appl. No.: 12/520,689

(22) PCT Filed: Dec. 21, 2007
(86) PCT No.: PCT/NL07/50692

§ 371 Date: Oct. 19, 2009

Related U.S. Application Data
(60) Provisional application No. 60/871,340, filed on Dec. 21, 2006.

(51) Int. Cl. G01N 33/92; C07H 15/10

(52) U.S. Cl. 435/29; 536/17.9

(57) ABSTRACT

The present invention is in the field of Fabry disease and concerns a pathogenic factor allowing diagnosis of Fabry disease. In particular lyso-ceramide trihexosamide (lyso-CTH) has been found to function as a diagnostic marker for Fabry disease.

### FIELD OF THE INVENTION

The present invention is in the field of Fabry disease and concerns a pathogenic factor allowing diagnosis of Fabry disease.

### BACKGROUND OF THE INVENTION

Fabry disease is one of several genetically inherited diseases called lysosomal storage disorders. It causes a wide range of signs and symptoms that can range from mild to severe and life threatening. Fabry disease, also known as angiokeratoma corporis diffusum universale, Morbus Fabry, and Anderson-Fabry disease, is a progressive, X-chromosome-linked genetic disorder resulting from a defect in the gene for the lysosomal enzyme alpha-galactosidase A (alpha-GAL). This enzyme deficiency results in an accumulation of glycosphingolipids, particularly globotriaosylceramide (also abbreviated as Gb3, GL-3, or ceramide trihexosamide (CTH)), in the vascular endothelium and visceral tissues throughout the body. Because clinical presentation is widely variable and symptoms may mimic those of other diseases, diagnosis of Fabry disease is often overlooked or delayed. Despite being an X-linked disorder, some females may express varying degrees of clinical manifestations. Moreover, there are variants of Fabry disease that do not present with classical signs and symptoms. This suggests that the actual incidence of Fabry disease may be higher than currently estimated incidence of 1 in 40,000 males (panethnic).

Since Fabry is X-linked, the disease predominantly affects males (hemizygotes), who have little if any endogenous alpha-GAL. Although X-linked recessive diseases generally do not affect females, there are female carriers (heterozygotes) who may experience varying degrees of disease manifestations. It is believed that X-chromosomal inactivation (lyonization), which can block expression of the functional alpha-GAL gene in all or some parts of the body, is responsible for disease onset in carriers. Although the prevalence of female carriers who develop overt clinical manifestations is unknown, recent studies indicate that manifestations in carrier females are more common than previously thought.

The inability to catabolize GL-3 leads to progressive multisystemic damage to the kidney, heart, and cerebrovascular system. The clinical course of Fabry disease is usually marked by chronic pain, angiokeratomas, hypohidrosis, heat and cold intolerance, corneal opacities, renal failure, stroke, and cardiac complications. As the disease progresses, complications may become life-threatening. Progressive organ and tissue damage associated with Fabry disease may result in substantially decreased life expectancy. Before the availability of renal dialysis or transplantation, the average age of death among patients with classical Fabry disease was 41 years; today, average life expectancy is still only 50 years.

Signs and symptoms associated with Fabry disease are widely varied, making diagnosis challenging. Clinical onset usually occurs in childhood or adolescence, but symptoms are frequently misinterpreted or overlooked. Accurate diagnosis is frequently not established until adulthood, when the disease has progressed, and organ dysfunction or failure has occurred.

The cardinal presenting features of Fabry disease are intermittent acroparesthesia and episodic crises of pain and fever (especially in childhood), angiokeratomas, hypohidrosis, heat and cold intolerance, and a characteristic “whorled” corneal opacity that does not affect vision. Progressive accumulation of GL-3 in the vascular endothelium and other tissues leads to life-threatening manifestations in adulthood involving the heart, kidneys, central and peripheral nervous system, and cerebrovascular system.

Below an overview is provided of the signs and symptoms of Fabry disease that may be seen at different stages of life. Both male and female patients may experience some or all of these manifestations to varying degrees, depending in part on the extent of alpha-GAL activity levels.

Childhood: episodic pain crises, acroparesthesia; hypohidrosis; corneal and lenticular opacities; recurrent fever; heat and cold intolerance.

Adolescence: gastrointestinal manifestations; angiokeratomas; fatigue; episodic pain crises, acroparesthesia; hypohidrosis; corneal and lenticular opacities; recurrent fever; heat and cold intolerance.

Adulthood: renal insufficiency/failure; neurological complications; cerebrovascular disease; cardiac dysfunction; hearing loss and tinnitus; gastrointestinal manifestations; angiokeratomas; fatigue; episodic pain crises, acroparesthesia; hypohidrosis; corneal and lenticular opacities; recurrent fever; heat and cold intolerance.

Growing evidence indicates there may be a significant number of “atypical variants”-hemizygotes who have few or none of the hallmark symptoms of classical Fabry disease. Atypical variants have residual plasma alpha-GAL levels (1% to 30% of normal) and present much later in life than patients with classical Fabry disease. They are often identified serendipitously, and usually have manifestations predominately in one organ system.

Clinical heterogeneity and the rarity of Fabry disease makes diagnosing Fabry disease a challenge. The age of presentation, presenting symptoms, and clinical course vary from individual to individual. Greater recognition of Fabry disease symptoms may lead to earlier suspicion and diagnosis, which in turn may result in more effective disease management.

Any of the symptoms described above may lead to a presumptive clinical diagnosis of Fabry disease. However, because Fabry disease is a multisystemic disorder, patients may present different symptoms to a wide range of specialists. One confounding factor in diagnosis is the fact that many common signs and symptoms of Fabry disease are misattributable to other conditions.

Once a presumptive diagnosis of Fabry disease has been made based on clinical signs and symptoms, definitive diagnosis can be made by testing for deficient alpha-gal enzyme activity in plasma, leukocytes, tears, or biopsied tissue.

Although females carrying the Fabry gene may be asymptomatic or present with mild clinical manifestations, definitive identification of carriers is important. Diagnosis allows practitioners to monitor for new or worsening symptoms, and can help with identifying other family members with the disease.

Affected females can be diagnosed with Fabry disease by very low or absent alpha-GAL activity and by lipid deposition in biopsied tissues or urinary sediment. Many female carriers (with or without symptoms) have below-normal levels of alpha-GAL activity and/or the characteristic corneal opacities. However, this is not true for all carriers—some have alpha-GAL activity in the low to normal range. In families with an identified mutation, mutation analysis is the definitive way to identify carrier females. In families for whom a specific mutation is not documented, linkage analysis can be performed to establish carrier status.

Disease management strategies may include medications and lifestyle approaches to symptom relief and interventions to delay serious sequelae due to organ damage (eg, kidney transplantation, cardiac pacemaker insertion).

While symptom management may improve a patient's quality of life, treatment to prevent or reverse accumulation of GL-3 and offers the potential to stem disease progression and prevent organ damage.

Gene therapy for Fabry disease is in the early stages of investigation. Research has identified a couple of different approaches that show potential promise in pre-clinical studies in Fabry mice. Research has also identified two approaches involving “small molecules.” Both of these require some residual alpha-GAL activity to be effective and could potentially be used in conjunction with either gene therapy or enzyme replacement therapy. The first approach involves substrate inhibition therapy to reduce cellular synthesis of glycosphingolipids. Two potentially promising small molecules are N-butyldeoxynojirimycin and D-threo-1-ethylendioxyphenyl-2-palmitoylamino-3-pyrrolidino-propanol (D-t-EtDO-P4). A second approach involves use of a competitive inhibitor, in particular 1-deoxy-galactonojirinmycin, of alpha-GAL to increase the activity of residual enzyme.

Enzyme replacement therapy (ERT) is currently available in the United States and in over 27 additional countries for people with Fabry disease. Following the success of enzyme replacement therapy for type 1 Gaucher disease, comparable therapies have been developed for the treatment of Fabry disease. Chronic intravenous administration of the registered recombinant alpha-galactosidase preparations (agalsidase alpha (Replagal®, Shire) and agalsidase beta (Fabrazyme®, Genzyme)) aims to correct the alpha-galactosidase A deficiency in cells of Fabry patients and thus to reverse, or at least stop, storage material accumulation in lysosomes and the accompanying pathological processes. Unfortunately it has become clear that clinical responses to enzyme replacement therapies in Fabry patients are far less spectacular than those shown by Gaucher patients receiving a comparable intervention.

To improve the efficacy of enzyme replacement therapies for Fabry disease additional insight is clearly needed regarding the impact of the common formation of (neutralizing) antibodies directed against the therapeutic enzymes in male Fabry patients. Needed is better insight regarding optimal enzyme dosing regimens for individual Fabry patients. Furthermore, the most appropriate time of therapeutic intervention has to be established since it is found that the clinical impact of therapeutic intervention is considerably poorer in patients with already established extensive disease.

### DESCRIPTION OF THE INVENTION

It is so far believed that the pathogenesis of Fabry disease simply mimics that of other lysosomal disorders, for example type 1 Gaucher disease. The envisioned sequence of events is thus as follows. Alpha-galactosidase A deficiency causes accumulation of its corresponding substrate (the globoside Gb3). The lysosomal storage somehow results in cellular dysfunction and damage. This ongoing process finally leads to organ failures manifesting as clinical symptoms. There are several compelling indications that in reality the pathogenesis of Fabry disease is far more complex. Firstly, a considerable number of female Fabry heterozygotes develop a severe course of disease, closely resembling that of male Fabry hemizygotes completely lacking alpha-galactosidase A. The presence of alpha-galactosidase A competent cells and circulating enzyme in female heterozygotes apparently hardly prevents disease onset and progression. This phenomenon differs markedly from the situation in Hunter disease, another X-linked lysosomal storage disorder. Secondly, although most male Fabry hemizygotes completely lack alpha-galactosidase A, disease manifestation occurs nevertheless relatively late in life. Again this markedly differs from any other lysosomal storage disorder for which complete lack of degradation capacity is either incompatible with life or causes infantile phenotypes. Thirdly, genetically engineered Fabry mice quickly develop pronounced Gb3 storage in the endothelium but not the characteristic organ failures of Fabry patients. Apparently there is not a very close relation between primary Gb3 accumulation and pathogenic processes. It has to be concluded from the discussion above that some crucial link is missing in our present understanding of the pathogenesis of Fabry disease.

It is an object of the present invention to trace the crucial missing link in the pathogenesis of Fabry disease. It is an object of the present invention to find a way to improve the diagnosis of Fabry disease.

Upon extensively researching lipid profiles in Fabry patients, the present inventor has surprisingly found that that lyso-ceramide trihexosamide (lyso-CTH) is dramatically elevated in plasma of Fabry patients. Lyso-CTH is formed as side-product from ceramide trihexosamide (CTH), either by ceramidase or protease activity. Lyso-CTH is a potent inhibitor of both alpha-galactosidase A and B. Up to now there are no parameters that predict pathogenesis of Fabry disease and response to therapies. With the finding of the aberrant plasma-levels of lyso-CTH in Fabry patients a unique diagnostic tool for Fabry disease is provided. Thus, monitoring of lyso-CTH will offer a completely new tool that can actively guide clinicians in clinical decision making regarding start of (preventive) treatment and individualized dosing regimens for Fabry disease.

With this finding also an explanation of how Fabry disease generally can remain sub-clinical for almost one decade, where after the pathophysiological processes seem to accelerate in the third decade in male and the fourth decade in females. Firstly alpha-galactosidase B can partly eliminate alpha-galactosidase A deficiency. In other words, alpha-galactosidase B displays the same activity as alpha-galactosidase A towards Gb3, be it with a much lower Km towards the substrate. However, the gradual generation and release of a side product of the Gb3 storage material can inhibit alpha-galactosidase A and B. In deed it was confirmed that lyso-CTH inhibits both alpha-galactosidase A and B. This explains the late manifestation of Fabry disease and the comparable course of the disorder in males (virtually) lacking alpha-galactosidase A and females with competent cells and circulating alpha-galactosidase A protein.

With this finding, lyso-CTH itself also is identified as toxic component in Fabry disease. Lyso-CTH was found to promote in vitro smooth muscle cell proliferation at concentrations as occurring in plasma of Fabry patients. Smooth muscle cell proliferation leads to vascular aberrations and cardiac hypertrophy. Having now identified lyso-CTH as the key pathogenic factor in Fabry disease, levels in plasma of this pathogen need to be reduced to come to a successful therapy.

Based on the identification of lyso-CTH as a pathogenic factor in Fabry disease the following sequence of events in Fabry disease is postulated:
1 primary CTH storage (very early in males)
2 slow formation of lyso-CTH
3 lyso-CTH starts to inhibit alpha-galactosidase A and B
4 lyso-CTH exerts its deleterious effects

In particular the present invention concerns lyso-CTH as a diagnostic marker for Fabry disease. In one embodiment the present invention relates to a method for diagnosing Fabry disease, said method comprising measuring the concentration of lyso-ceramide trihexosamide (lyso-CTH) in a plasma sample of a subject. In another embodiment the invention comprises further the step of comparing the concentration of lyso-CTH measured in a plasma sample of a subject with a standard concentration of lyso-CTH. It is advantageous to compare the concentration of lyso-CTH measured in a plasma sample of a subject with a standard concentration of lyso-CTH which is the average concentration of lyso-CTH in plasma samples of individuals that are known to have no deficiency of lysosomal enzyme alpha-galactosidase A.

In view of the fact that Fabry disease is an X-linked disorders it is particularly advantageous to discriminate in the present diagnostic method between males and females. In one embodiment the present method is advantageous to diagnose Fabry disease in males. In an other embodiment the present method is advantageous to diagnose Fabry disease in females. Further it is advantageous that if the subject for which the present method is carried out is male, the results are compared with the average concentration of lyso-CTH in plasma samples of individuals that are also male. In turn it is advantageous that if the subject for which the present method is carried out is female, the results are compared with the average concentration of lyso-CTH in plasma samples of individuals that are also female.

In particular it was found in males that the concentration of lyso-CTH was increased >50 fold in plasma of Fabry patients compared to standard concentrations. This same increase can also ascribed to females, at least to adolescent and adult females, as lyso-CTH concentrations in the range of pmol/ml were found whereas in standard samples, if present at all, concentrations are below pmol/ml.

Thus the present invention also concerns a diagnostic marker for Fabry disease, said diagnostic marker being the concentration of lyso-CTH in a plasma sample of a subject. In one embodiment the concentration of lyso-CTH is >50 times the concentration of lyso-CTH in individuals that are known to have no deficiency of lysosomal enzyme alpha-galactosidase A.

Methods of measuring the concentration of lyso-CTH in plasma samples are available and known to those skilled in the art. For example measuring the concentration of lyso-CTH in plasma samples involves Bligh and Dyer extraction preferably followed by butanol/water extraction. Next the extracted lysosphingolipids, including Iso-CTH, may be derivatised with a label in order to facilitate detection. Analysis can be routinely carried out on a HPLC system preferably equipped with a reversed phase column. Preferably an internal standard is included in order to properly quantify the results obtained, i.e. measure the actual concentration of lyso-CTH in the plasma sample of the subject.

Alternatively HPLC-tandem MS can be used to analysis lyso-CTH in plasma samples. For example the simultaneous quantitative analysis of bioactive sphingolipids by high-performance liquid chromatography-tandem mass spectrometry is described by Bielawski et al in Methods. 2006 June; 39(2):82-91 which is incorporated herein by reference. In general there has been a recent explosion in research concerning novel bioactive sphingolipids (SPLs) such as ceramide (Cer), sphingosine (Sph) and sphingosine 1-phosphate (Sph-1P) that necessitates development of accurate and user-friendly methodology for analyzing and quantitating the endogenous levels of these molecules. ESI/MS/MS methodology provides a universal tool used for detecting and monitoring changes in SPL levels and composition from biological materials. Simultaneous ESI/MS/MS analysis of sphingoid bases (SBs), sphingoid base 1-phosphates (SB-1Ps), Cers and sphingomyelins (SMs) is performed on a Thermo Finnigan TSQ 7000 triple quadrupole mass spectrometer operating in a multiple reaction monitoring (MRM) positive ionization mode. Biological materials (cells, tissues or physiological fluids) are fortified with internal standards (ISs), extracted into a one-phase neutral organic solvent system, and analyzed by a Surveyor/TSQ 7000 LC/MS system. Qualitative analysis of SPLs is performed by a Parent Ion scan of a common fragment ion characteristic for a particular class of SPLs. Quantitative analysis is based on calibration curves generated by spiking an artificial matrix with known amounts of target synthetic standards and an equal amount of IS. The calibration curves are constructed by plotting the peak area ratios of analyte to the respective IS against concentration using a linear regression model. This robust analytical procedure can determine the composition of endogenous sphingolipids (ESPLs) in varied biological materials and achieve a detection limit at 1 pmol or lower level. This and related methodology are already defining unexpected specialization and specificity in the metabolism and function of distinct subspecies of individual bioactive SPLs.

The present invention also relates to a kit of parts for diagnosis of Fabry disease, said kit of parts comprising at least one selected form the group consisting of butanol, means for Bligh and Dyer extraction, labels for purpose of detection, lyso-CTH as a standard, Gb3 as a standard, a reference lysosphingolipid and instructions for carrying out an analysis of lyso-CTH.

Also the present invention relates to a method of monitoring and or a method of optimizing a therapy for Fabry disease, said method comprising measuring the concentration of lyso-ceramide trihexosamide (lyso-CTH) in a plasma sample of a subject. Preferably in these methods the concentration that is measured is compared with a standard concentration as described above. Alternatively in these methods the concentration is measured after a therapeutic intervention and preferably is compared with a standard concentration as described above or is compared with the concentration of plasma lyso-CTH in the same subject prior to the therapeutic intervention. Optimisation of dosage, dosage form, route of administration, nature of therapeutic agent etc can be achieved in this way.

### EXAMPLES

### Analysis

The formula below represent the structures of Gb3 (CTH) and lyso-CTH (lyso-Gb3)

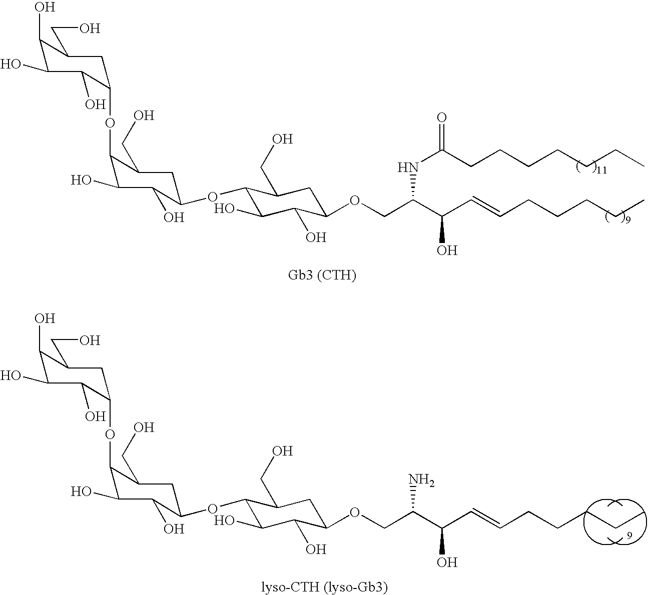

An optimal extraction procedure for lyso-CTH from plasma samples was established. A double extraction was carried out, first a Bligh and Dyer extraction followed by butanol extraction.

Partitioning of lyso-CTH (% of total)

| | upper phase | lower phase |
|---|---|---|
| Bligh and Dyer | 90 | 10 |
| butanol/water | 99 | 2 |

The concentration of lyso-CTH was measured as follows:

Plasma samples were extracted by the procedure of Bligh and Dyer. The upper phase was dried under N2 and subjected to butanol/water extraction. The upper phase was dried under N₂ and the residue was taken up in 250 pt methanol.

The residue, including lysosphingolipids, dissolved in methanol were derivatised on line for 30 min with o-phtalaldehyde. Analysis was performed using an HPLC system (Waters Associates, Milford, Mass.) and a Hypersil BDS C18 3μ, 150×4.6 mm reverse phase column (Alltech). Chromatographic profiles were analysed using Waters Millenium software. The eluent used was methanol:water; 88:12 (w/w).

By standard addition of lyso-CTH to normal plasma a calibration curve was constructed.

The tables below show the results of a number of samples that were analysed.

TABLE 1
Lyso-CTH in plasma of male members of family H

| Age | Genetic status | lyso-CTH (pmol/ml) |
|---|---|---|
| 50 | normal | 2 |
| 24 | normal | 3 |
| 20 | hemizygote | 253 |
| 18 | hemizygote | 166 |
| 16 | hemizygote | 315 |
| 11 | hemizygote | 483 |
| 9 | normal | 0 |

Thus in male Fabry patients, or males with a predisposition to become Fabry patients, a dramatically increase of plasma lyso-CTH is found. Plasma lyso-CTH in all tested symptomatic males>50 fold normal mean.

TABLE 2
Lyso-CTH in plasma of female members of family H

| Age | Genetic status | lyso-CTH (pmol/ml) |
|---|---|---|
| 45 | heterozygote | 83 |
| 42 | heterozygote | 19 |
| 17 | normal | 0 |
| 15 | heterozygote | 21 |
| 8 | normal | 0 |
| 7 | heterozygote | 0 |
| 5 | normal | 0 |

Thus starting form at least adolescence in female Fabry patients, or females with a predisposition to become Fabry patients, a dramatically increase of plasma lyso-CTH is found with concentration in the pmol/ml range whereas normal mean values are sub pmol/ml.

### Toxicity

The toxic effect of lyso-CTH was determined in vitro in SMC-41 cell line. ³H thymidine incorporation (per 24 h) was determined as a function of lyso-CTH concentration. At concentrations of lyso-CTH as found in plasma of Fabry patients, i.e. 0.065-0.65 μM, increased isotope incorporation and increased proliferation of SMC-41 was found

1. A method for diagnosing Fabry disease, comprising
(a) measuring the concentration of lyso-ceramide trihexosamide (lyso-CTH) in a plasma sample of a subject;
(b) comparing the concentration of said lyso-CTH with a standard concentration of lyso-CTH,
wherein an elevated concentration in the subject's sample compared to the standard concentration is indicative of Fabry disease.

2. (canceled)

3. The method according to claim 1 wherein the standard concentration is the average concentration of lyso-CTH in plasma samples of control individuals who are known not to be deficient in lysosomal enzyme alpha-galactosidase A.

4. (canceled)

5. A method of optimizing treatment for Fabry disease, comprising
(a) measuring the concentration of lyso-CTH in a plasma sample of a subject with Fabry disease before treatment; and
(b) measuring the concentration of lyso-CTH after treatment with a selected therapeutic agent administered at a selected dose, in a selected dosage form, and/or by a selected route,
wherein a decrease in said lyso-CTH concentration after said treatment indicates that the treatment with the selected agent, dose, dosage form and/or route of administration is optimized compared to a different agent, dose, dosage form or route.

6. The method according to claim 5 wherein the lyso-CTH concentration in a plasma sample from said subject after said therapeutic intervention is compared to the average concentration of lyso-CTH in plasma samples of control individuals who are known not to be deficient in lysosomal enzyme alpha-galactosidase A.

7. The method according to claim 13 wherein the subject and the control individuals are male.

8. The method according to claim 13, wherein the subject and the control individuals are female.

9. (canceled)

10. A diagnostic marker for Fabry disease, comprising the presence of an elevated concentration of lyso-CTH in a plasma sample of a subject with, or suspected of having, Fabry disease compared to the lyso-CTH concentration in control individuals who are known not to be deficient in lysosomal enzyme alpha galactosidase A.

11. The diagnostic marker according to claim 10 wherein the elevated lyso-CTH concentration is >50 times the concentration of lyso-CTH in said control individuals.

12. The method according to claim 1 wherein said lyso-CTH in said subject's sample is at least about 50-fold higher than said standard concentration.

13. The method according to claim 3 wherein the sex of the subject and the control individuals is the same.

14. The method according to claim 6 wherein the sex of the subject and the control individuals is the same.

15. The method according to claim 14, wherein the subject and the control individuals are male.

16. The method according to claim 14, wherein the subject and the control individuals are female.
